(19) United States
(12) Patent Application Publication
(10) Pub. No.: US 2009/0281112 A1
(43) Pub. Date: Nov. 12, 2009

(54) ADATANSERIN AND METABOLITES THEREOF FOR TREATMENT OF ATTENTION DEFICIT DISORDER, ANXIETY, DEPRESSION, SEXUAL DYSFUNCTION, AND OTHER DISORDERS

(75) Inventors: Stephen J. KRAMER, Houston, TX (US); Louis F. FABRE, Houston, TX (US)

Correspondence Address: OBLON, SPIVAK, MCCLELLAND MAIER & NEUSTADT, L.L.P., 1940 DUKE STREET, ALEXANDRIA, VA, 22314, US

(73) Assignee: Fabre-Kramer Pharmaceuticals, Inc., Houston, TX (US)

(21) Appl. No.: 12/437,245

(22) Filed: May 7, 2009

Related U.S. Application Data
(60) Provisional application No. 61/051,540, filed on May 8, 2008.

(51) Int. Cl. A61K 31/496; A61K 31/4965; A61K 31/506; A61P 25/00

(52) U.S. Cl. 514/252.18; 514/255.01; 514/252.19; 514/254.11

(57) ABSTRACT

The present invention relates to a method for alleviation, prevention, and treatment of attention deficit disorder, anxiety, depression, sexual dysfunction, aggressive behavior, alcohol dependency, psychoses, and related conditions by administering adatanserin and/or metabolites of adatanserin. The present invention also relates to a method of weight loss by administering adatanserin and/or metabolites of adatanserin.

### BACKGROUND OF THE INVENTION

1. Field of the Invention

The present invention relates to a method for alleviation, prevention, and treatment of attention deficit disorder, anxiety, depression, sexual dysfunction, aggressive behavior, alcohol dependency, psychoses, and related conditions by administering adatanserin and/or metabolites of adatanserin. The present invention also relates to a method of weight loss by administering adatanserin and/or metabolites of adatanserin.

2. Discussion of the Background

### Attention Deficit Disorder

Attention deficit disorder (ADD) is a learning disorder that relates to developmentally inappropriate inattention and impulsivity. ADD may also be referred to as disruptive behavior disorder or minimal brain dysfunction. ADD may be present with or without hyperactivity. A common disorder, ADD accounts for more child mental health referrals than any other single disorder. ADD is estimated to affect 3 to 5% of school-aged children, and is much more frequent in males than in females, with a male-to-female ratios ranging from 4:1 to 9:1. On the average, at least one child in every classroom in the United States needs help for the disorder. ADD often continues into adolescence and adulthood, and can cause a lifetime of frustrated dreams and emotional pain. In addition, ADD may affect the behavior of children at any cognitive level.

ADD is a diagnosis applied to children and adults who consistently display certain characteristic behaviors over a period of time. The most common behaviors fall into two categories: inattention and impulsivity. Attention deficit disorder with hyperactivity is diagnosed when the signs of overactivity are obvious. Inappropriate inattention causes increased rates of activity and impersistence or reluctance to participate or respond. A subject suffering from ADD exhibits a consistent pattern of inattention and/or hyperactivity-impulsivity that is more frequent and severe than is typically observed in individuals at a comparable level of development. Such subjects must suffer clear evidence of interference with developmentally appropriate social, academic, or occupational functioning.

Although subjects with ADD and without hyperactivity may not manifest high activity levels, most exhibit restlessness or jitteriness, short attention span, and poor impulse control. These are qualitatively different from those seen in conduct and anxiety disorders. Inattention is described as a failure to finish tasks started, easy distractibility, seeming lack of attention, and difficulty concentrating on tasks requiring sustained attention. Impulsivity is described as acting before thinking, difficulty taking turns, problems organizing work, and constant shifting from one activity to another. Impulsive responses are especially likely when involved with uncertainty and the need to attend carefully. Hyperactivity is featured as difficulty staying seated and sitting still, and running or climbing excessively. A more complete description of the symptoms and diagnostic criteria of attention deficit disorder with or without hyperactivity are provided by DSM-IV (Diagnostic and Statistical Manual of Mental Disorders, 1994; 78-85), which is incorporated herein by reference.

No single treatment has been completely effective for attention deficit disorder. Psychostimulant medications combined with behavioral and cognitive therapies (e.g., selfrecording, self-monitoring, modeling, and role-playing) have the greatest controlling influence on symptom expression. Used alone, medication has been effective predominantly with less aggressive ADD children coming from stable home environments. Elimination diets, megavitamin treatments, psychotherapy, and biochemical interventions (e.g., the administration of neurochemicals) have had only minor, unsustained effects.

For decades, medications have been used to treat the symptoms of ADD. The three most common medications in both adults and children are the stimulants; methylphenidate (RITALIN™), dextroamphetamine (DEXEDRINE™ or DEXTROSTAT™), and pemoline (CYLERT™). For many people, these medicines dramatically reduce their hyperactivity and improve their ability to focus, work, and learn. The medications may also improve physical coordination, such as handwriting and ability in sports. Recent research by National Institute of Mental Health suggests that these medicines may also help children with an accompanying conduct disorder to control their impulsive, destructive behaviors. Nine out of 10 children improve on one of these three stimulant drugs.

Different doctors use the medications in slightly different ways. CYLERT™ is available in one form, which naturally lasts 5 to 10 hours. RITALIN™ and DEXEDRINE™ come in short-term tablets that last about 3 hours, as well as longer-term preparations that last through the school day.

Stimulant drugs, when used with medical supervision, are usually considered safe. However, a common problem with stimulant drugs is that they can be addictive to teenagers and adults if misused. While on these medications, some children may lose weight, have less appetite, and temporarily grow more slowly. Others may have problems falling asleep. Some doctors believe that stimulants may also make the symptoms of Tourette's syndrome worse.

The most commonly prescribed ADD medication is RITALIN™, which is generally more effective than tricyclic antidepressants (e.g., IMIPRAMINE™), caffeine, and other psychostimulants (e.g., PEMOLINE™ and DEANOL™) and has fewer side effects than dextroamphetamine. Common side effects of RITALIN™ are sleep disturbances (e.g., insonmia), depression or sadness, headache, stomachache, suppression of appetite, elevated blood pressure, and, with large continuous doses, a reduction of growth. Long-term benefits of medication with RITALIN™, however, have not been demonstrated conclusively. Some research indicates that use of medication permits participation in activities previously inaccessible because of poor attention and impulsivity. The frequency of side effects, potential addictiveness, and limited success of stimulant drugs has led to a search for alternate means of treating or preventing attention deficit disorders.

Anxiety

Anxiety is a condition characterized by cognitive, physical, emotional, and behavioral components, combining to result in feelings typically recognized as fear, apprehension, or worry. Anxiety is often accompanied by physical sensations such as heart palpitations, nausea, chest pain, shortness of breath, stomach aches, or headache. The cognitive component entails recognition of a threat of certain danger. Physically, the body prepares the organism to deal with such a threat, including increased blood pressure, heart rate, perspiration, and blood circulation to major muscle groups. The body also experiences inhibition of the immune and digestive system functions. External signs of anxiety may include pale skin, sweating, trembling, or pupillary dilation. Emotionally, anxiety causes a sense of dread or panic and physically causes nausea, diarrhea, and chills. Behaviorally, both voluntary and involuntary behaviors may arise directed at escaping or avoiding the source of anxiety. Anxiety is not always pathological. Rather, it is a common emotion along with fear, anger, sadness, and happiness, with an important function in relation to survival. However, anxiety can be maladaptive, with the most extreme cases being expressed as anxiety disorders.

Anxiety can be classified by its severity. Severe anxiety disorders only affects a small minority of anxiety sufferers, and may include severe panic and anxiety disorders like obsessive-compulsive disorder (OCD), post-traumatic stress disorder (PTSD), social phobias, and stress disorders. Severe anxiety disorders are highly treatable but require medical diagnosis. Generalized anxiety disorder, characterized as by mild to moderate anxiety, is far more common but harder to identify. Generalized anxiety disorder is characterized by compulsive worrying and physical symptoms of anxiety which persist for more than six months.

The causes of anxiety are not completely understood. It is likely caused by an interrelationship between multiple factors. It is known that a neurotransmitter imbalance can sensitize a person's brain to experiencing anxious feelings or fear. Consistently high levels of excitatory neurotransmitters, such as norepinephrine and epinephrine, along with low levels of inhibitory neurotransmitters, such as serotonin and gamma-aminobutyric acid, may result in predisposition to anxiety disorders. Caffeine is known to result in increased levels of dopamine, and sufficient amounts may result in anxiety. The neurotransmitter imbalances that cause anxiety are related to those in children with ADHD and ADD, which are associated with higher levels of dopamine.

In addition to neurotransmitter imbalances, imbalances in the hypothalamic-pituitary-adrenal (HPA) axis can influence anxiety. The hypothalamus releases corticotropin-releasing factor (CRF) as part of the cascade of hormones in the fight-or-flight response. CRF stimulates adrenocorticotropic hormone (ACTH), which in turn acts on the adrenal glands, which release cortisol. In normal situations, cortisol allows for the rush of glucose, fat, and protein that gives cells increased energy and alertness. In healthy individuals, the hormonal release recedes once the perceived threat is addressed. Disruption of the HPA axis, however, can result in persistently increased levels of ACTH and cortisol, resulting in anxiety. Other hormones, including sex hormones, may affect anxiety. For example, higher levels of estrogen, along with lower levels of progesterone, may result in decreased serotonin levels.

Although acute attacks of anxiety are not experienced by every anxiety sufferer, they are a common symptom. Anxiety attacks usually come without warning, and although the fear is generally irrational, the perceived danger is very real. A person experiencing an anxiety attack will often feel as if they are about to die or pass out, may experience fear, and may feel the need to avoid certain stressful situations or social situations due to fear of embarrassment. The person may experience considerable confusion and irritability when experiencing anxiety. Physical symptoms include hot flushes, chest pain, sudden exhaustion, headaches, and shortness of breath, digestive problems, and nausea.

Anxiety may be treated with medication, therapy, or a combination of both. Other treatment modalities may include self-help techniques, lifestyle changes, and alternative medicine for relief and wellness. Medications used for treatment of anxiety include antidepressants. These may include selective serotonin reuptake inhibitors, tricyclic antidepressants, and monoamine oxidase inhibitors. Other antianxiety drugs include benzodiazepines and buspirone. While these may be effective, the side effects of such treatments can be significant, including the development of dependency or addiction. The frequency of side effects, potential addictiveness, and limited success of stimulant drugs has led to a search for alternate means of treating or preventing attention deficit disorders.

Depression

Depression is a psychiatric disorder, characterized by a pervasive low mood, loss of interest in usual activities and diminished ability to experience pleasure. Clinical depression affects about 8-17% of the population on at least one occasion in their lives, before the age of forty. In the United States, it has been estimated that approximately 14 million adults suffer depression per year. Although the term “depression” is commonly used to describe a temporary condition when one “feels blue”, clinical depression is a serious and often disabling condition that can significantly affect a person's work, family and school life, sleeping and eating habits, general health and ability to enjoy life. The course of clinical depression varies widely. Depression can be a once in a life-time event or have multiple recurrences. It can appear either gradually or suddenly, and either last for a few months or be a life-long disorder. Depression is a major risk factor for suicide, and people with depression suffer from higher mortality due to other causes of death.

Clinical depression may be a primary condition, or it may be secondary to another condition, such as chronic pain. Clinical depression can present with a variety of symptoms. Most patients display a marked change in mood, a deep feeling of sadness, and a noticeable loss of interest or pleasure in favorite activities. Other symptoms include persistent sadness or anxiety, loss of appetite and/or weight loss, increased appetite and overeating and weight gain, insomnia, restlessness or irritability. The depressed patient may experience feelings of worthlessness, inappropriate guilt, helplessness, hopelessness, and/or pessimism. He may have difficulty thinking, concentrating, remembering or making decisions. The depressed patient may have suicidal thoughts, and may attempt suicide. Decreased energy, fatigue, and sluggishness, along with persistent physical symptoms that do not respond to treatment, such as headaches, digestive problems, and chronic pain, are often experienced by depressed patients. Diagnosis of depression is detailed in Diagnostic and Statistical Manual of Mental Disorders (Fourth Ed.) (American Psychiatric Association (1994), herein incorporated by reference in its entirety.

There are numerous theories about causes of depression. It is generally believed that clinical depression is caused by an influence of some combination of biological and genetic factors, environmental influences, and childhood or developmental events. Several risk factors have been identified. In the United States, women are about as twice as likely as men to be diagnosed and treated for major depression. People over the age of 65 may be especially vulnerable. Previous episodes increase the risk of depression, as approximately one-half of those who have developed depression will experience it again. And people who have relatives who have had clinical depression have a greater chance of developing it themselves, suggesting a genetic component.

The biological causes of clinical depression may include disordered brain function, neurotransmitters, and hormones. Researchers studying clinical depression tend to look at several aspects of brain function including the structures of the limbic system and the function of neurotransmitters within neurons. Researchers have identified associations between clinical depression and the function of serotonin, norepinephrine, and dopamine. These three neurotransmitters function within structures of the brain that regulate emotions, reactions to stress, and the physical drives of sleep, appetite, and sexuality.

Neurotransmitters play a role in the development or treatment of clinical depression. It has been shown that many people who are depressed have low levels of the neurotransmitter norepinephrine. The use of some antidepressants can increase the level of norepinephrine in the brain, and subsequently relieve depressive symptoms. However, some depressed people have high levels of norepinephrine, making the causation link unclear.

The dopamine system is an important factor in depression. Short-term surges of dopamine are normally associated with feelings of pleasure. However, abnormally high concentrations of the chemical messenger may be an important cause of depression.

The endocrine system is also involved in the causation of clinical depression. The hormones that are released into the body by the endocrine glands regulate processes such as reaction to stress and sexual development. A great number of depressed people have abnormal levels of some hormones in their blood despite having healthy glands, which is believed to result in problems with appetite and sleeping. Problems with hormone levels may be intertwined with the changes in brain chemistry that are seen in clinical depression. The endocrine system is connected with the brain at the hypothalamus which controls many bodily activities such as sleep, appetite, and sexual drive. The hypothalamus also regulates the pituitary gland that, in turn, controls the hormonal secretion of other glands. The hypothalamus uses some of the neurotransmitters that have been associated with depression as it manages the endocrine system. These neurotransmitters, serotonin, norepinephrine, and dopamine all have a role in the management of hormone function.

Genetic factors and environmental factors, such as prolonged stress at home or work, coping with the loss of a loved one, or traumatic events, may also contribute to depression.

Sexual Dysfunction

Sexual Dysfunction may be defined as difficulty during any stage of the sexual act (which includes desire, arousal, orgasm, and resolution) that prevents the individual or couple from enjoying sexual activity. Sexual dysfunction disorders are generally classified into four categories: sexual desire disorders, sexual arousal disorders, orgasm disorders, and sexual pain disorders.

Sexual desire disorders (decreased libido) may be caused by a decrease in the normal production of estrogen (in women) or testosterone (in both men and women). Other causes may be aging, fatigue, pregnancy, and medications (such as anti-depressants such as fluoxetine, sertraline, and paroxetine are well known for reducing desire in both men and women. Psychiatric conditions, such as depression and anxiety, can also cause decreased libido.

Sexual arousal disorders were previously known as frigidity in women and impotence in men. These have now been replaced with less judgmental terms. Impotence is now known as erectile dysfunction, and frigidity is now described as any of several specific problems with desire, arousal, or anxiety. For both men and women, these conditions may appear as an aversion to, and avoidance of, sexual contact with a partner. In men, there may be partial or complete failure to attain or maintain an erection, or a lack of sexual excitement and pleasure in sexual activity.

Orgasm disorders are a persistent delay or absence of orgasm following a normal sexual excitement phase. The disorder occurs in both women and men. Again, the SSRI antidepressants are frequent culprits—these may delay the achievement of orgasm or eliminate it entirely.

Sexual pain disorders affect women almost exclusively, and are known as dyspareunia (painful intercourse) and vaginismus (an involuntary spasm of the muscles of the vaginal wall, which interferes with intercourse). Dyspareunia may be caused by insufficient lubrication (vaginal dryness) in women.

Sexual dysfunctions are more common in the early adult years, with the majority of people seeking care for such conditions during their late 20s through 30s. The incidence increases again in the geriatric population, typically with gradual onset of symptoms that are associated most commonly with medical causes of sexual dysfunction. Sexual dysfunction is more common in people who abuse alcohol and drugs. It is also more likely in people suffering from diabetes and degenerative neurological disorders. Ongoing psychological problems, difficulty maintaining relationships, or chronic disharmony with the current sexual partner may also interfere with sexual function.

Symptoms of sexual dysfunction may include loss of libido, inability to feel aroused, painful intercourse in both male and female patients. In men, symptoms may include inability to attain or maintain an erection, delay or absence of ejaculation, and inability to control timing of ejaculation. In women, symptoms may include inability to relax vaginal muscles enough to allow intercourse, inadequate vaginal lubrication before and during intercourse, inability to attain orgasm, and burning pain on the vulva or in the vagina with contact to those areas.

Sexual dysfunction is common among individuals with depression. Depressed individuals show decreased sexual interest and reported reduced levels of arousal. Sexual dysfunction is also a common side effect of antidepressant treatment, particularly pharmacotherapy with serotonin reuptake inhibitors (SRIs). The sexual response cycle consists of 4 phases: desire, arousal, orgasm, and resolution. All of these phases may be affected by reproductive hormones and neurotransmitters. Estrogen, testosterone, and progesterone promote sexual desire. Dopamine promotes desire and arousal. Norepinephrine promotes arousal. Prolactin inhibits arousal. Oxytocin promotes orgasm. Serotonin may have a negative impact on the desire and arousal phases of the sexual response cycle, possibly due to its inhibition of dopamine and norepinephrine.

Treatment of sexual dysfunction involves identifying the specific cause and, often, treating the underlying condition. Medical causes that are reversible or treatable are usually managed medically or surgically. Physical therapy and mechanical aides may prove helpful for some people experiencing sexual dysfunction due to physical illnesses, conditions, or disabilities.

Neurological and psychological factors play an important role in sexual dysfunction. Anxiety, fear, and depression can particularly be addressed with treatments such as psychological therapy and medications. Dopamine is known to promote desire and arousal. Accordingly, dopaminergic agents may be helpful for the treatment of antidepressant-induced sexual dysfunction.

Psychoses

Psychosis is a general term for a mental state described as involving a loss of contact with reality. People experiencing psychoses may report hallucinations or delusional beliefs, and may exhibit personality changes and disorganized thinking. This may be accompanied by unusual or bizarre behaviour, as well as difficulty with social interaction and impairment in carrying out the activities of daily living.

The signs and symptoms of psychoses include one or more of hallucinations, delusions, thought disorder, and lack of insight. Hallucinations are defined as sensory perception in the absence of external stimuli. Hallucinations may occur in any of the five senses and take on almost any form, which may include simple sensations (such as lights, colors, tastes, and smells) to more meaningful experiences such as seeing and interacting with fully formed animals and people, hearing voices and complex tactile sensations. Auditory hallucinations, particularly the experience of hearing voices, are a common and often prominent feature of psychosis. Delusions are typically beliefs which are sometimes paranoid in nature. Psychotic delusions have been classified as primary or secondary. Primary delusions are defined as having no known source and not being comprehensible in terms of normal mental processes. Secondary delusions may be understood as being influenced by the person's background or situation, such as ethnicity, sexuality, religion, or superstition. Thought disorder is an underlying disturbance to conscious thought. It is classified largely by its effects on speech and writing, with affected persons often showing speech pressures (speaking incessantly and quickly), derailment or flight of ideas (switching topic mid-sentence or inappropriately), thought blocking, and rhyming or punning. Lack of insight is generally defined as the psychotics lack of awareness of the unusual, strange, or bizarre nature of his experience or behavior.

Psychoses are classified descriptively based on behavioral and clinical observations. This approach is adopted in the standard guide to psychiatric diagnoses employed in the United States, the Diagnostic and Statistical Manual of Mental Disorders (DSM), incorporated herein by reference. The DSM lists nine psychoses, or formal psychotic disorders. The formal psychotic disorders are: schizophrenia, schizoaffective disorder, schizophreniform disorder, brief psychotic disorder, delusional, shared psychotic disorder, substance induced disorder, psychosis due to a general medical condition, and psychosis—not otherwise specified.

The DSM classifies psychotic disorders as traditional psychotic illnesses, psychosis due to general medical conditions, and substance induced psychosis. Traditional psychoses may be related to schizophrenia, bipolar disorder (manic depression), severe clinical depression, severe psychosocial stress, and sleep deprivation. Psychosis arising from “organic” (non-psychological) can be associated with the neurological disorders, electrolyte disorders, hypoglycemia, lupus, AIDS, leprosy, malaria, and other conditions. Neurological disorders that can give rise to psychoses may include brain tumours, multiple sclerosis, sarcoidosis, lyme disease, syphilis, Alzheimer's Disease, and Parkinson's Disease. Electrolyte disorders may include hypocalcemia, hypematremia, hyponatremia, hypokalemia, hypomagnesemia, hypermagnesemia, hypercalcemia, and hypophosphatemia. Psychoses may also be caused by ingestion of many legal and illegal substances, including alcohol, dextromethorphan, certain antihistamines, cold medications including phenylpropanolamine, barbiturates, benzodiazepines, isotretinoin, anticholinergic drugs (such as atropine, scopolamine, and Jimson weed), antidepressants, L-dopa, antiepileptics, stimulants (including cocaine, amphetamines, methamphetamine, methylphenidate, MDMA (ecstasy)) hallucinogens (including cannabis, psilocybin, mescaline, and PCP), psychotropics (including LSD).

The pathophysiology of psychoses is the subject of much study. Brain imaging studies of psychosis, investigating both changes in brain structure and changes in brain function of people undergoing psychotic episodes, have shown significant structural changes in the brains of some people with psychosis, including grey matter reduction in the cortex of people before and after they became psychotic. Functional brain scans have revealed that the areas of the brain that react to sensory perceptions are active during psychosis. For example, a PET or MRI scan of a person who claims to be hearing voices may show activation in the auditory cortex, or parts of the brain involved in the perception and understanding of speech. One consistent finding is that persons with a tendency to have psychotic experiences seem to show increased activation in the right hemisphere of the brain. The significance of this finding is not well-understood.

Psychosis have been linked to the neurotransmitter dopamine. The dopamine hypothesis of psychosis states that psychosis results from an overactivity of dopamine function in the brain, particularly in the mesolimbic pathway. This is based largely on the fact that dopamine-blocking drugs tend to reduce the intensity of psychotic symptoms, and that drugs which boost dopamine activity (such as amphetamine and cocaine) can trigger psychosis in some people. However, the connection between dopamine and psychosis is generally believed to be complex.

Treatment of psychosis depends on the cause or diagnosis. Antipsychotic medications and psychological therapy are frequently used. Other treatments, such as electroconvulsive therapy have also been utilized. Based on the link to the dopaminergic system, dopaminergic agents may be helpful for the treatment of psychoses.

Aggressive Behavior

Aggressive behavior is behavior that is intended to cause harm or pain. It can be either physical or verbal. To distinguish, behavior that accidentally causes harm or pain is not aggression, and aggression is not synonymous with assertiveness. Aggression is currently categorized broadly as either (1) hostile, affective, or retaliatory aggression, or as (2) instrumental, predatory, or goal-oriented aggression. Some evidence suggests that the two types are psychologically and physiologically different. For example, some research indicates that people with tendencies toward affective aggression have lower IQs than those with tendencies toward predatory aggression. There are also thought to be gender-based differences.

The biology of aggressive behavior is complex. Aggression is directed to and often originates from outside stimuli, but has a very distinct internal character. Within the brain, the amygdala has been shown to be critically involved in aggression, as stimulation of the amygdala results in augmented aggressive behavior. Further, lesions of this area greatly reduce aggression. In addition, the hypothalamus serves a regulatory role in aggression. The hypothalamus has been shown to cause aggressive behavior when electrically stimulated and expresses receptors that help determine aggression levels based on their interactions with the neurotransmitters serotonin and vasopressin. The prefrontal cortex is also associated with the regulation of aggression. Reduced activity of the prefrontal cortex, in particular its medial and orbitofrontal portions, has been associated with violent/antisocial aggression. Specifically, regulation of the levels of the neurotransmitter serotonin in the prefrontal complex has been connected with a particular type of pathological aggression.

Neurotransmitters and hormones are correlated with aggressive behavior. The concentration of testosterone is closely correlated with aggressive responses, at least as measured in several studies of the concentration of blood testosterone of convicted male criminals who committed violent crimes compared to males without a criminal record or who committed non-aggressive crimes. Further, testosterone levels in female criminals versus females without a criminal record mirror those of males: testosterone levels are higher in women who commit aggressive crimes or are deemed aggressive by their peers than non-aggressive females.

Cortisone also plays a role in regulating aggressive behavior. In adult rats, injections of corticosterone promote aggressive behavior and reduction of corticosterone decreases aggression. Paradoxically, a chronically low level of corticosterone can produce abnormally aggressive behavior.

The neurotransmitter serotonin has been shown to be related to aggression, which may explain the aggression-reducing effects of selective serotonin reuptake inhibitors such as fluoxetine. Indeed, certain types of aaggressive behavior in laboratory animals, such as that relying on social instigation, frustrative non-reward and alcohol drinking, are modulated by dopamine, serotonin (5-HT) and GABA. The dopamine system is implicated in the initiation, execution, termination and consequences of aggressive behavior.

Because the dopamine system is linked to aggressive behavior, dopaminergic drugs are thought to be helpful. However, there remains a need for dopaminergic drugs with a high affinity for dopamine receptors that may reduce aggressive behavior.

### Alcohol Dependence

Alcohol Dependence, or alcoholism, is a condition characterized by the harmful consequences of repeated alcohol use, a pattern of compulsive alcohol use, and physiological dependence on alcohol. The physiological dependence is characterized by development of tolerance to increased levels of alcohol and symptoms of withdrawal upon discontinuation of abuse. Alcohol dependence is diagnosed when these behaviors become persistent, disabling, or distressing.

Persons suffering from alcoholism face many complications. School and job performance may suffer. Child care or household responsibilities may be neglected. Absences from school or job may occur. The individual may use alcohol in physically hazardous circumstances, such as driving or operating machinery while intoxicated. Moreover, legal difficulties may arise because of alcohol use, including arrests for intoxicated behavior or for drunk driving. Alcohol intoxication causes significant intellectual impairment.

Physical problems seen with alcohol abuse may include depression, blackouts, liver disease, and other complications. Nearly every organ system is effected. Gastrointestinal effects include gastritis, stomach or duodenal ulcers, liver cirrhosis and pancreatitis. There is also an increased rate of cancer of the esophagus, stomach, and other parts of the gastrointestinal tract. Cardiovascular effects include hypertension, along with an elevated risk of heart disease. Nervous system effects include peripheral neuropathy, as evidenced by muscular weakness, paresthesis, and decreased peripheral sensation. Central nervous system effects include cognitive deficits, severe memory impairment, and degenerative changes in the cerebellum, resulting in poor balance and coordination. Individuals with this disorder are at increased risk for accidents, violence, and suicide. Severe Alcohol Intoxication also contributes to disinhibition and feelings of sadness and irritability, which contribute to suicide attempts and completed suicides.

Individuals suffering from alcoholism continue to abuse alcohol despite knowing that doing so causes significant social or interpersonal problems for them. Alcoholics may devote substantial time, effort, and financial resources to obtaining and consuming alcohol.

Individuals with Alcohol Dependence are at increased risk for Major Depressive Disorder, other Substance-Related Disorders (e.g., drug addiction), Conduct Disorder in adolescents, Antisocial and Emotionally Unstable (Borderline) Personality Disorders, Schizophrenia, and Bipolar Disorder.

The causes of alcohol dependence are not well-understood. However, it is clearly a disorder strongly determined by cultural and psychological factors. On the other hand, a genetic susceptibility seems clear. Alcohol Dependence often has a familial pattern, and it is estimated that 40%-60% of the variance of risk is explained by genetic influences. The risk for Alcohol Dependence is 3 to 4 times higher in close relatives of people with Alcohol Dependence. Studies have found a significantly higher risk for Alcohol Dependence in the monozygotic twin than in the dizygotic twin of a person with Alcohol Dependence. Adoption studies have revealed a 3- to 4-fold increase in risk for Alcohol Dependence in the children of individuals with Alcohol Dependence when these children were adopted away at birth and raised by adoptive parents who did not have this disorder.

The familial pattern of alcohol dependence suggests a genetic basis. Among the possible genetic causes are genes related to the dopamine D2 receptor. Blum and co-workers (32) found this marker more often in alcoholics than in nonalcoholics. See Blum et al, Allelic association of human dopamine D2 receptor gene in alcoholism, Journal of the American Medical Association 263(15):2055-2060, 1990, and Comings et al, The dopamine D2 receptor locus as a modifying gene in neuropsychiatric disorders, Journal of the American Medical Association 266(13):1793-1800, 1991, both herein incorporated by reference in their entirety. In animal studies, the dopamine D2 receptor had been associated with brain functions relating to reward, reinforcement, and motivation. Some researchers believe dopamine D2 might modulate the severity of alcoholism, rather than serve as a primary cause.

The dopamine D2 association continues to be interesting, but it does not seem to be transmitted in families in such a way that it is responsible for alcoholism; its role, if any, has yet to be determined. However, present animal and human data suggest several alterations in the dopaminergic system in the context of alcoholism. Receptor studies imply that dominergic D₂ receptor density and function are lower at least among type 1 alcoholics, suggests that they could benefit from drugs that enhance dopaminergic activity, such as partial dopaminergic agonists.

### Weight Loss

Obesity is a condition in which the natural energy reserve, stored in the fatty tissue of humans and other mammals, exceeds healthy limits. It is commonly defined as a body mass index (weight divided by height squared) of 30 kg/m² or higher. Although obesity is an individual clinical condition, some authorities view it as a serious and growing public health problem. Some studies show that excessive body weight has been shown to predispose to various diseases, particularly cardiovascular diseases, diabetes mellitus type 2, sleep apnea and osteoarthritis.

Obesity can be classified in absolute or relative terms using measurements such as body mass index (BMI). BMI is calculated with the following formula:

Metric:BMI=kg/m²

where kg is the subject's weight in kilograms and m is the subject's height in meters. The BMI can be used to classify, as follows, using definitions established by the World Health Organization:
A BMI less than 18.5 is underweight
A BMI of 18.5-24.9 is normal weight
A BMI of 25.0-29.9 is overweight
A BMI of 30.0-39.9 is obese
A BMI of 40.0 or higher is severely (or morbidly) obese
A BMI of 35.0 or higher in the presence of at least one other significant comorbidity is also classified by some bodies as morbid obesity.
   Waist: Hip ratios, waist circumference, and body fat measurement are also used to assess obesity.

A large number of medical conditions have been associated with obesity. Health consequences include those associated with increased fat mass (osteoarthritis, obstructive sleep apnea, social stigma) or increased number of fat cells (diabetes, cancer, cardiovascular disease, non-alcoholic fatty liver disease). Mortality is increased in obesity, with a BMI of over 32 being associated with a doubled risk of death. Moreover, alterations in the body's response to insulin), a proinflammatory state, and an increased tendency to thrombosis (prothrombotic state), are seen in obese patients.

Most researchers have concluded that the combination of an excessive nutrient intake and a sedentary lifestyle are the main cause for obesity. Less well established and possibly underinvestigated life style issues that may influence obesity include (1) insufficient sleep, (2) endocrine disruptors—food substances that interfere with lipid metabolism, (3) decreased variability in ambient temperature, (4) decreased rates of smoking, which suppresses appetite, (5) increased use of medication that leads to weight gain, (6) increased distribution of ethnic and age groups that tend to be heavier, (7) pregnancy at a later age, (8) intrauterine and intergenerational effects, (9) positive natural selection of people with a higher BMI, (10) assortative mating, heavier people tending to form relationships with each other.

As with many medical conditions, the caloric imbalance that results in obesity is probably the result of a combination of genetic and environmental factors. Certain people may be predisposed to obesity. Certain physical and mental illnesses and particular pharmaceutical substances may predispose to obesity. Apart from the fact that correcting these situations may improve the obesity, the presence of increased body weight may complicate the management of others. Medical illnesses that increase obesity risk include several rare congenital syndromes (listed above), hypothyroidism, Cushing's syndrome, growth hormone deficiency. Smoking cessation is a known cause for moderate weight gain, as nicotine suppresses appetite. Medications, such as steroids, anti-psychotics, and fertility drugs may cause weight gain. Mental illnesses may also increase obesity risk, specifically some eating disorders such as bulimia nervosa, binge eating disorder, and compulsive overeating (also known as food addiction).

Obesity tends to run in families, and overeating seems linked to other family-related disorders such as alcoholism and drug addiction. All appear to involve disruptions to the brain's reward system, which dispenses pleasure-inducing dopamine in response to species-propagating behavior such as eating, drinking water or having sex. Cocaine addicts, for example, have fewer dopamine receptors either as a result of the constant stimulation by the drug—it blocks transporters that normally usher dopamine back into brain cells to be recycled—or because they were born that way. Overeaters have a similar shortage of dopamine receptors

Body weight is controlled mostly by the hypothalamus. The hypothalamus reacts to ghrelin, a hormone excreted by an empty stomach, by releasing appetite-stimulating neurotransmitters into other parts of the brain. It shuts off the neurotransmitters in response to leptin, a hormone released by fat cells. Dopamine is believed to play a role in the communication of the hypothalamus with the cerebral cortex, the site of conscious thought. Drugs that stimulate dopamine release within the hypothalamus may reduce appetite. One possible mechanism is as follows:

(1) Empty stomach releases ghrelin, an appetite stimulant, to the hypo-thalamus, which controls the body's metabolism.
(2) The hypothalamus releases dopamine to the nucleus accumbens and striatum, motivating the conscious area of the brain to find food.
(3) The smell of food stimulates the amygdala, also a center of emotion, and causes further dopamine release by the nucleus accumbens.
(4) Sight, smell and taste of food stimulates release of endorphins (opioids) and dopamine by the orbitofrontal cortex, further stimulating the conscious part of the brain to eat.
(5) Leptin released by fat cells eventually overpowers ghrelin and signals the hypothalamus to shut down the dopamine flow. As a result, appetite wanes.

Treatment for obesity is generally by eating fewer calories and exercising more. However, not all dieters are successful. Moreover, it has proven difficult for dieters to maintain their weight-loss long term. Behavior modification and counseling have proven useful.

In cases where exercise, intake reduction, and behavior modification are insufficient, pharmacotherapy can be offered. Sibutramine, which blocks the reuptake of the neurotransmitters dopamine, norepinephrine, and serotonin, can be helpful. Orlistatem which blocks the absorption of fat, is used. Phentermine stimulates neuron bundles to release a neurotransmitters including dopamine, epinephrine, and norepinephrine. Other drugs that act on the dopaminergic system that may be helpful in treating obesity include diethylpropion, fluoxetine, and bupropion.

There remains a need for more effective dopaminergic therapy for weight loss.

Therapy for the treatment of ADD, anxiety, depression, psychoses, aggressive behavior, alcohol dependence, obesity, and/or sexual dysfunction may be to target the dopaminergic system. Deregulation of the dopaminergic system has been linked with each of these conditions. Such deregulation has also been linked with Parkinson's disease, Tourette's syndrome, schizophrenia, attention deficit hyperactive disorder (ADHD) and generation of pituitary tumours (Vallone et al, Neurosci Biobehav Rev 2000 January; 24:125-32). The azapirone, buspirone, has been speculated as having a potential therapeutic role in treatment of ADHD (Balon, J. Clin. Psychopharma. 1990; 10: 77, and Malhotra et al, J. Am. Acad. Child Adolesc. Psychiatry 1998; 57: 364-371).

Buspirone exhibits an affinity for a series of receptors including serotonin receptors, dopamine receptors, and α-adrenergic receptors. The effect of buspirone on the dopaminergic system occurs by enhancing dopamine synthesis and release (Tunnicliff et al, Neuropharmacology 1992; 31: 991-5). Buspirone blocks the presynaptic dopaminergic receptors rather than the postsynaptic dopaminergic receptors, thereby increasing the firing of the midbrain neurons and blocking the inhibiting effects of γ-aminobutyric acid on dopaminergic neurons in the zona compacta of the substantia nigra (Eison and Temple, Am. J. Med. 1986; 80(3B suppl): 1-9). Although the serotonergic activity may be related to improved behavior and impulsivity, according to Balon and Malhotra et al, the dopaminergic activity of buspirone leads to improved attention span and decreased hyperactivity associated with ADHD. However, more recent reports on treatment strategies of attention deficit disorder have suggested that buspirone may have a potentially deleterious effect on patients having ADHD (Popper, Child Adolesc Psychiatr Clin NAm 2000; 9: 605-46). Busprone may be expected to have similar deleterious effects on patients with other conditions linked to the dopaminergic system.

Accordingly, there remains a critical need for novel treatment strategies of patients suffering from ADD, with or without hyperactivity, anxiety, depression, psychoses, aggressive behavior, alcohol dependence, obesity, and sexual dysfunction. Moreover, there remains a critical need for treatment strategies, which are safe and effective with a reduction in or elimination of any side effects associated with existing treatment strategies.

### SUMMARY OF THE INVENTION

Accordingly, it is an object of the present invention to provide methods for treatment of attention deficit disorder, with or without hyperactivity, anxiety, depression, psychoses, aggressive behavior, alcohol dependence, obesity, and sexual dysfunction, or symptoms thereof. This object can be achieved by administering to a patient in need thereof with a 5-HT_{1A} receptor agonist.

The present invention is based, in part, on the discovery that a patient suffering from attention deficit disorder can be treated by an azapirone 5-HT_{1A} receptor agonist, which lacks dopamine receptor activity. Accordingly, it is an object of the present invention to treat a patient having attention deficit disorder, with or without hyperactivity, or symptoms thereof, with an azapirone 5-HT_{1A} receptor agonist, which lacks dopamine receptor activity. Examples of suggested azapirone 5-HT_{1A} receptor agonists, which lack dopamine receptor activity, include gepirone, ipsapirone, and tandospirone.

It is another object of the present invention to treat a patient having treatment of attention deficit disorder, with or without hyperactivity, anxiety, depression, and sexual dysfunction, or symptoms thereof with an adamantyl aryl- or heteroaryl piperazinyl carboxamide 5-HT_{1A} receptor agonist. One example of this 5-HT_{1A} receptor agonist is adatanserin.

It is another object of the present invention to treat a patient having attention deficit disorder, with or without hyperactivity, or symptoms thereof, with a hetrobicyclic-arylpiperazine 5-HT_{1A} receptor agonist. One example of this 5-HT_{1A} receptor agonist is flesinoxan.

In one embodiment, the 5-HT_{1A} receptor agonist is administered in conjunction with an agent selected from the group consisting of a stimulant, a hypnotic, an anxiolytic, an antipsychotic, an antianxiety agent, a minor tranquilizer, a benzodiazepine, a barbituate, a serotonin agonist, a selective serotonin reuptake inhibitor, a dopamine antagonist, a 5-HT_{1A} agonist, a 5-HT₂ antagonist, a non-steroidal anti-inflammatory drug, a monoamine oxidase inhibitor, a muscarinic agonist, a norephinephrine uptake inhibitor, an essential fatty acid, and a neurokinin-1 receptor antagonist.

In another embodiment, the 5-HT_{1A} receptor agonist is administered with the administration of methylphenidate (RITALIN™).

In another embodiment, the 5-HT_{1A} receptor agonist is administered with a pharmaceutically acceptable carrier.

In another embodiment, the 5-HT_{1A} receptor agonist is administered orally, rectally, nasally, parenterally, intracistemally, intravaginally, intraperitoneally, sublingually, topically, or bucally.

In another embodiment, the therapeutically effective amount of the 5-HT_{1A} receptor agonist is similar to the anxiolytic dose of the medication, e.g.: 0.25-0.75 mg/kg of body weight/day of gepirone (approximately 15 mg/day), 0.003-0.06 mg/kg of body weight/day of flesinoxan (approximately 0.4 mg/day), and 0.5-3.0 mg/kg of body weight/day of adatanserin (approximately 120 mg/day) in single or multiple doses.

In another embodiment, the total daily dose of including ipsapirone and tandospirone administered to a patient in need thereof, in single or in divided doses, can be in amounts of 0.25-3.0 mg/kg of body weight/day.

In another embodiment, the patient in need thereof also suffers from one or more of anxiety, depression, obesity, drug abuse/addiction, alcohol abuse, sleep disorders, TIC disorder, and behavioral/cognitive symptoms of Alzheimer's disease.

Another object of the present invention is to provide a treatment regimen of concurrently administering to a patient in need thereof mixtures of two or more of the compounds of the present invention.

Yet another object of the present invention is to provide a treatment regimen of administering to a patient in need thereof a single or divided dose of a first compound followed by, on the same day or a subsequent day, a single or divided dose of one or more additional compounds.

The above object highlights certain aspects of the invention. Additional objects, aspects and embodiments of the invention are found in the following detailed description of the invention.

### BRIEF DESCRIPTION OF THE DRAWINGS

A more complete appreciation of the invention and many of the attendant advantages thereof will be readily obtained as the same becomes better understood by reference to the following Figures in conjunction with the detailed description below.

FIG. 1: The 5-HT (serotonin) neuron in the hyperactive state: namely, high levels of 5-HT, overactive 5-HT neuronal firing and transmission, and down-regulated somatodendritic presynaptic 5-HT_{1A} autoreceptors.

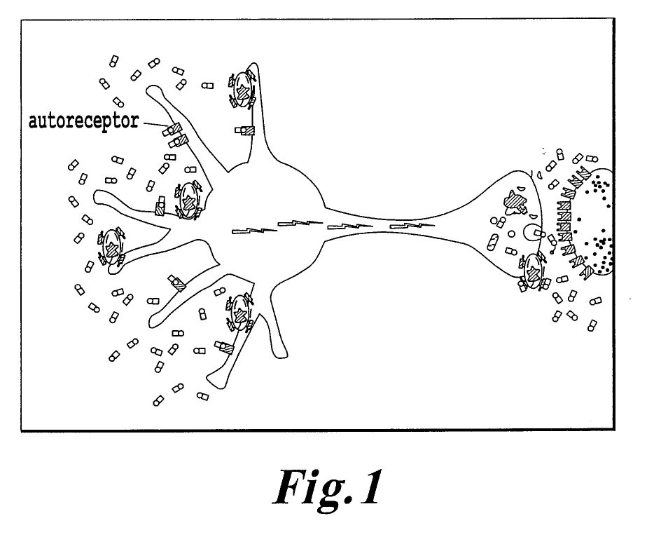

FIG. 2: The drugs (gepirone, ipsapirone, tandospirone, flesinoxan, and adatanserin) are presynaptic agonists and post-synaptic partial agonists. A presynaptic agonist shuts off the neuron (less serotonin post-synaptically). Post-synaptic partial agonism results in less robust stimulation of the post-synaptic neuron than serotonin itself (resulting in less firing). The net result is less post-synaptic neuron firing.

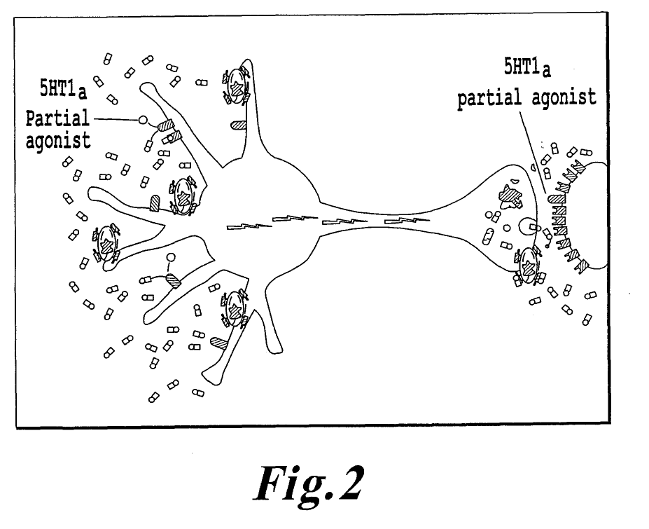

### DETAILED DESCRIPTION OF THE PREFERRED EMBODIMENTS

Unless specifically defined, all technical and scientific terms used herein have the same meaning as commonly understood by a skilled artisan in organic chemistry, biochemistry, psychology, psychiatry, medicine, neurochemistry, and neurology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified.

As used herein, the general term “attention deficit disorder” includes attention deficit disorder and disruptive behavior disorder each of which may be present with or without hyperactivity.

The term “5-HT_{1A} receptor agonist” means partial agonist or full agonist with respect to either presynaptic receptors or postsynaptic receptors, or both, unless otherwise specified.

In psychopharmacology, the serotonin (5-hydroxytryptamine [5-HT]) type-1A (5-HT_{1A}) receptor has acquired considerable attention as a therapeutic target. Agonism of the 5-HT_{1A} receptor has been correlated with treatment of anxiety, depression, obesity, drug abuse/addiction, alcohol abuse, sleep disorders and behavioral and cognitive symptoms of Alzheimer's disease (U.S. Pat. No. 4,423,049, U.S. Pat. No. 4,771,053, U.S. Pat. No. 5,106,849, and Bison, Journal of Clinical Psychopharma, 1990; 10: S2-S5). Accordingly, an embodiment of the present invention is to treat a patient suffering from attention deficit disorder and at least one of the above-stated disorders by administering a therapeutically effective amount of the compounds and/or preparations of the present invention. Further, an embodiment of the present invention is to treat anxiety, depression, and sexual dysfunction in patients suffering attention deficit disorder as well as those not suffering attention deficit disorder.

The use, preparation, and characterization of therapeutic azapirone compounds have been disclosed in numerous documents (see Cadieux, Amer. Family Physician 1996 53: 2349-2353; Temple, U.S. Pat. No. 4,423,049; Gawin, U.S. Pat. No. 5,185,329; Madding, U.S. Pat. No. 5,521,313). This class of compounds attributes its activity to partial agonism of the 5-HT_{1A} receptor.

Clinical studies of known 5-HT_{1A} agonists and partial agonists, for example buspirone, ipsapirone, and gepirone, have shown that these compounds are useful in the treatment of anxiety disorders, such as generalized anxiety disorder (GAD), panic disorder, and obsessive compulsive disorder (Glitz. D. A., Pohl R., Drugs 1991, 41:1 1; Cadieux, Amer. Family Physician 1996 53: 2349-2353). Clinical and preclinical evidence supports 5-HT_{1A} partial agonists for use in treating depression as well as impulse control disorders and alcohol abuse (van Hest, Psychopharm., 107: 474 (1992); Schipper et al, Human Psychopharm., 6: S53 (1991); Cervo et al, Eur. J. Pharm., 158: 53 (1988); Glitz, D. A., Pohl, R., Drugs, 41: 11 (1991)). Studies show that 5-HT_{1A} agonists and partial agonists inhibit isolation-induced aggression in male mammals, indicating that they can be used to treat aggression (Sanchez et al. Psychopharmacology, 1993, 110:53-59). Other studies indicate that 5-HT_{1A} receptors are important in the serotonergic modulation of haloperidol-induced catalepsy (Hicks, Life Science 1990, 47:1609) suggesting that 5-HT_{1A} agonists can be used to treat the deleterious side effects of conventional antipsychotic agents, such as haloperidol. Recent reports show that this is the case for side effects like tardive dyskinesias.

The present invention is based, in part, on the Inventor's surprising discovery that certain 5-HT_{1A} receptor agonists can be employed to treat attention deficit disorder, with or without hyperactivity, or symptoms thereof, and that certain 5-HT_{1A} receptor agonists can be employed to treat anxiety, depression, psychoses, aggressive behavior, alcohol dependence, obesity, and sexual dysfunction, or symptoms thereof. Specifically, this object can be achieved by administering to a patient in need thereof a therapeutically effective amount of a 5-HT_{1A} receptor agonist selected from an azapirone 5-HT_{1A} receptor agonist, which lacks dopamine receptor activity, a hetrobicyclic-aryl-piperazine 5-HT_{1A} receptor agonist, or an adamantyl aryl- or heteroaryl piperazinyl carboxamide 5-HT_{1A} receptor agonist.

In one embodiment, the azapirone 5-HT_{1A} receptor agonist, which lacks dopamine receptor activity, is gepirone. Gepirone (also known as 4,4-dimethyl-1-[4-[4-(2-pyrimidinyl)1-piperazinyl]-butyl]-2,6-piperadinedione hydrochloride) can be obtained by the process according to Temple (U.S. Pat. No. 4,423,049, which is incorporated herein in its entirety by reference) and has the following structure:

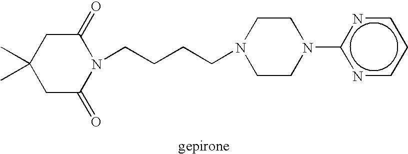

Briefly, a solution of 3,3-dimethylglutaric anhydride and 1-(4-aminobutyl)-4-(2-pyrimidinyl)piperazine in xylene is refluxed, collecting water of reaction by means of a Dean Stark trap. The reaction mixture is then filtered while warm (approximately 80° C.) and the filtrate concentrated in vacuo to give a residue. The residue is then distilled and the distillation product is either crystallized from acetonitrile to give a solid base, or is treated with ethanolic HCl to give the hydrochloride salt.

In other embodiments, ipsapirone (2-(4-(4-(2-pyrimidinyl)-1-piperazinyl)-butyl) 1,2-benzoisothiazol-3(2H)-one 1,1-dioxide hydrochloride) and tandospirone (N-[4-[4-(2-pyrimidinyl)-1-piperazinyl]butyl]-2,3-norbornanedicarboximide). Ipsapirone and methods of making ipsapirone are disclosed in U.S. Pat. No. 4,988,700 and Traber et al (Trends Pharmacol Sci 1987; 8: 432-7), both of which are incorporated herein by reference; tandospirone and methods of making tandospirone are disclosed in U.S. Pat. Nos. 4,507,303; 4,543,355; 4,598,078 and 5,011,841, all of which are incorporated herein by reference.

Methods of assessing the receptor (5-HT_{1A} and dopamine) agonistic activity of the compounds of the present invention are well known to those of skill in the art. These methods can be found in Iser-Strenegr, et al (Brain Res 1986 November; 395(t):57-65), Millan et al (J Pharmacol Exp Ther 1993 March; 264(3):1364-76), and Perrone R. et al (J Med Chem 1995 Mar. 17; 38(6):942-9), all of which are incorporated herein by reference.

Flesinoxan ((+)-N-[2-[4-(2,3-dihydro-2-hydroxymethyl-1,4-benzodioxin-5-yl)-lpiperaxinyl]ethyl]-4-fluorobenzamide hydrochloride) is a high affinity and high selectivity 5-HT_{1A} receptor agonist, which has been shown to have both anxiolytic and antidepressant activity (Hadrava et al, Neuropharmacology, 1995; 34(10): 1311-1326). Accordingly, in another embodiment of the present invention the 5-HT_{1A} receptor agonist that can be employed to treat attention deficit disorder, with or without hyperactivity, or symptoms thereof, is the hetrobicyclic-aryl-piperazine 5-HT_{1A} receptor agonists. Examples of other suitable compounds of this class, as well as methods of making these compounds, are disclosed in Hartog et al (U.S. Pat. No. 4,833,142, which is incorporated herein by reference).

In another embodiment of the present invention, a patient having attention deficit disorder, with or without hyperactivity, or symptoms thereof, can be administered an adamantyl aryl- or heteroaryl piperazinyl carboxamide 5-HT_{1A} receptor agonist to treat the disorder. As a specific example of this class of compounds, adatanserin (N-[2-[4-(2-pyrimidinyl)-1-piperazinyl]ethyl]tricyclo[3.3.1.1^{3,7}]decane-1-carboxamide) is mentioned. Adamantyl aryl- or heteroaryl piperazinyl carboxamide 5-HT_{1A} receptor agonist compounds envisioned as suitable for administration in accordance with this invention can be synthesized as described by Abou-Gharbia, et al (U.S. Pat. Nos. 5,106,849; 5,010,078; 5,380,725; 5,482,940; 5,278,160; 5,254,552; and J Med Chem 1999 Dec. 16; 42(25):5077-94, all of which are incorporated herein by reference).

A drug that treats attention deficit disorder is assessed by measuring the child's behavior before and after treatment with a drug of the present invention. Measurements of the child's behavior include clinical measures and rating scales. Two clinical measures are the simulated classroom (Gadow et al, Stony Brook, N.Y.: Checkmate Plus, 1996) and the continuous performance test (Roberts et al, J Pediatr Psychol 1984; 9:177-191, Halperin et al, J Am Acad Child Adolesc Psychiatry 1992; 31:190-196, and Halperin et al, J Am Acad Child Adolesc Psychiatry 1988; 27:326-329).

The simulated classroom requires the child to sit alone at a desk in a small classroom completing work, and not playing with toys on an adjacent table. Clinic sessions are video-recorded through a one-way window to facilitate ease of scoring. The 3 ADHD behaviors measured are Off Task, Fidgeting, and Worksheets (number of items completed correctly). The continuous performance test (CPT) requires a child to press the space bar whenever the letter “A” followed the letter “X” on a computer screen. The CPT generates 3 scores (inattention, impulsivity, and dyscontrol) and takes approximately 12 minutes to complete. Examples of rating scales include the Abbreviated Teacher Questionnaire (ATQ; Conners, Psychopharm Bull 1973; 9:24-84 and Epstein et al, J Special Educ 1986; 20:219-229), the Iowa-Corners Teachers Rating Scale (Loney et al, Advances in developmental and behavioral pediatrics 1982; vol. 3, Greenwich, Conn.:JAI Press; 113-147), and the Primary Secondary Symptom Checklist (Loney, Poster presented at the annual meeting of the American Psychological Association, Toronto, Ontario, 1984). Normally both teachers and parents scales are rated.

The results of the pre-treatment and post-treatment evaluations can be analyzed by appropriate statistical procedures, such as those contained in Mandel, The Statistical Analysis of Experimental Data, Dover Publications; Toronto, Ontario, 1964.

Based on the Inventor's surprising discovery it can be surmised that psychostimulants, such as methylphenidate (RITALIN™) work because they stimulate inhibitory pathways, whereas the serotonin partial agonists of the present invention work because they inhibit stimulatory pathways.

One skilled in the art is familiar with numerous methods for designing and optimizing formulations and delivery methods to deliver 5-HT_{1A} receptor agonists, in particular gepirone, ipsapirone, tandospirone, flesinoxan, and adatanserin in effective and non-toxic ways. Remington's Pharmaceuticals Sciences, 18th Edition (specifically incorporated herein by reference), can be relied on and used for these purposes, especially Part 8 therein, “Pharmaceutical Preparations and Their Manufacture.” The following compounds, compositions, delivery methods, delivery dosages, and formulations are specifically envisioned as suitable for, but not meant to limit, the present invention.

The pharmaceutical compounds suitable for administration in the present invention may be hydrochloride salts, but the free bases and other pharmaceutically acceptable salts are also suitable. The term “pharmaceutically acceptable salt” is well known in the art, as described in S. M. Berge et al. (J Pharmaceutical Sciences, 66: 1-19, 1977). Suitable pharmaceutically acceptable salts for administration in the present invention include acid addition salts. The acid addition salt may be formed by mixing a solution of the compound with a solution of a pharmaceutically acceptable non-toxic acid such as hydrochloric acid, hydrobromic acid, fumaric acid, maleic acid, succinic acid, acetic acid, citric acid, tartaric acid, carbonic acid, phosphoric acid, perchloric acid, sulphuric acid, oxalic acid, or malonic acid. Where the compound carries an acidic group, for example a carboxylic acid group, the present invention also contemplates salts thereof, preferably nontoxic pharmaceutically acceptable salts thereof, such as the sodium, potassium and calcium salts thereof.

Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pictate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, salts of amine groups. Salts of amine groups may also comprise the quaternary ammonium salts in which the amino nitrogen atom carries an alkyl, alkenyl, alkynyl or aralkyl group, nontoxic ammonium, quaternary ammonium, and amine cations formed using counterions such as halide, hydroxide, carboxylate, sulfate, phosphate, nitrate, loweralkyl sulfonate and aryl sulfonate.

A therapeutically effective amount of the pharmaceutical compounds suitable for administration in the present invention may be administered alone or in combination with one or more pharmaceutically acceptable carriers. As used herein, the term “pharmaceutically acceptable carrier” means a non-toxic, inert solid, semi-solid or liquid filer, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions suitable for administration in the invention can be administered to patients in need thereof orally, rectally, nasally, parenterally (e.g., intramuscular, intraperitoneal, intravenous or subcutaneous injection, or implant), intracisternally, intravaginally, intraperitoneally, sublingually, topically (e.g., as a powder, ointment, or drop), bucally, as an oral spray, or a nasal spray. The pharmaceutical compositions can be formulated in dosage forms appropriate for each route of administration.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art. The inert diluents may include, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. The liquid dosage form for oral administration may also contain adjuvants, which include wetting agents, emulsifying and suspending agents, sweetening, flavoring, and perfuming agents. Other dosage forms for oral administration include, for example, aqueous suspensions containing the active compound in an aqueous medium in the presence of a non-toxic suspending agent such as sodium carboxy-methylcellulose, and oily suspensions containing a compound of the present invention in a suitable vegetable oil, for example arachis oil.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This maybe accomplished by the use of a liquid suspension of crystalline or amorphous material with poor water solubility. The rate of absorption of the drug then depends upon its rate of dissolution, which, in turn, may depend upon crystal size and crystalline form. Alternatively, dissolving or suspending the drug in an oil vehicle accomplishes delayed absorption of a parenterally administered drug form. Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, prills, powders, and granules. In such solid dosage forms, the active compound is mixed with at least one inert, pharmaceutically acceptable excipient or carrier. In addition, the solid dosage form may contain one or more fillers, extenders, binders, humectants, disintegrating agents, retarding agents, absorption accelerators, wetting agents, absorbents, or lubricants. Examples of suitable fillers or extenders include, starches, lactose, sucrose, glucose, mannitol, and silicic acid, sodium citrate and dicalcium phosphate. Examples of suitable binders include, microcrystalline cellulose, carboxymethylcellulose, alginates, gelatin, polyvinylpyrrolidinone, sucrose, and acacia. Glycerol is an example of a suitable humectant. Examples of suitable disintegrating agents include, agar-agar, calcium carbonate, potato or tapioca starch, maize starch, alginic acid, certain silicates, and sodium carbonate. Paraffin is an example of a suitable solution-retarding agent. As absorption accelerators, any quaternary ammonium compound may be used. Examples of suitable wetting agents include, cetyl alcohol and glycerol monostearate. Examples of suitable absorbents include, kaolin and bentonite clay. Examples of suitable lubricants include, talc, calcium stearate, magnesium stearate, solid polyethylene glycols, sodium lauryl sulfate. In the case of capsules, tablets and pills, the dosage form may also comprise buffering agents.

The tablets may, if desired, be coated using known methods and excipients that may include enteric coating using for example hydroxypropylmethylcellulose phthalate. The tablets may be formulated in a manner known to those skilled in the art so as to give a sustained release of the compounds of the present invention. Such tablets may, if desired, be provided with enteric coatings by known methods, for example by the use of cellulose acetate phthalate. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Similarly, capsules, for example hard or soft gelatin capsules, containing the active compound with or without added excipients, may be prepared by known methods and, if desired, provided with enteric coatings in a known manner. The contents of the capsule may be formulated using known methods so as to give sustained release of the active compound. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Solid compositions of a similar type may also be employed as fillers in soft and hardfilled gelatin capsules using such excipients as lactose or milk sugar as well as high molecular weight polyethylene glycols.

If desired, the compounds of the present invention can be incorporated into slow release or targeted delivery systems such as polymer matrices, liposomes and microspheres. They may be sterilized, for example, by filtration through a bacteria-retaining filter, or by incorporating sterilizing agents in the form of sterile solid compositions that can dissolve in sterile water, or some other sterile injectable medium immediately before use.

The active compound may be formulated into granules with or without additional excipients. The granules may be ingested directly by the patient or they may be added to a suitable liquid carrier (for example, water) before ingestion. The granules may contain disintegrates, e.g. an effervescent couple formed from an acid and a carbonate or bicarbonate salt to facilitate dispersion in the liquid medium.

Dosage forms for topical or transdermal administration of a compound of this invention include ointments, pastes, creams, lotions, gels, powders, solutions, sprays, inhalants or patches. Transdermal patches have the added advantage of providing controlled delivery of a compound to the body. The rate can be controlled by either providing a rate controlling membrane or by dispersing the compound in a polymer matrix or gel. The active component is admixed under sterile conditions with a pharmaceutically acceptable carrier and any needed preservatives or buffers as may be required. Dissolving or dispensing the compound in the proper medium can make such dosage forms. Absorption enhancers can also be used to increase the flux of the compound across the skin. Ophthalmic formulation, eardrops, eye ointments, powders and solutions are also contemplated as being within the scope of this invention.

Dosage forms for topical administration may comprise a matrix in which the pharmacologically active compounds of the present invention are dispersed so that the compounds are held in contact with the skin in order to administer the compounds transdermally. A suitable transdermal composition may be prepared by mixing the pharmaceutically active compound with a topical vehicle, such as animal and vegetable fats, oils, petrolatum, waxes, paraffins, starch, tragacanth, cellulose derivatives, polyethylene glycols, silicones, bentonites, silicic acid, talc and zinc oxide, or mixtures thereof, together with a potential transdermal accelerant such as dimethyl sulphoxide or propylene glycol. Alternatively the active compounds may be dispersed in a pharmaceutically acceptable paste, cream, gel or ointment base. The amount of active compound contained in a topical formulation should be such that a therapeutically effective amount of the compound is delivered during the period of time for which the topical formulation is intended to be on the skin.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons. The therapeutically active compound may be formulated into a composition, which is dispersed as an aerosol into the patient's oral or nasal cavity. Such aerosols may be administered from a pump pack or from a pressurized pack containing a volatile propellant.

The therapeutically active compounds used in the method of the present invention may also be administered by continuous infusion either from an external source, for example by intravenous infusion or from a source of the compound placed within the body. Internal sources include implanted reservoirs containing the compound to be infused which is continuously released for example by osmosis and implants which may be (a) liquid such as an oily suspension of the compound to be infused for example in the form of a very sparingly water-soluble derivative such as a dodecanoate salt or a lipophilic ester or (b) solid in the form of an implanted support, for example of a synthetic resin or waxy material, for the compound to be infused. The support may be a single body containing the entire compound or a series of several bodies each containing part of the compound to be delivered. The amount of active compound present in an internal source should be such that a therapeutically effective amount of the compound is delivered over a long period of time.

It will be known to those skilled in the art that there are numerous compounds, which may be used for treating attention deficit disorder, anxiety, depression, or sexual dysfunction in a patient. Combinations of these therapeutic agents, some of which have also been mentioned herein, will bring additional, complementary, and often synergistic properties to enhance the desirable properties of these various therapeutic agents. In these combinations, the 5-HT_{1A} agonist and the therapeutic agents may be independently present in dose ranges from one one-hundredth to one times the dose levels which are effective when these compounds are used singly. In such combination therapy, the 5-HT_{1A} agonist may be administered with the other therapeutic agent (e.g., concurrently, concomitantly, sequentially, or in a unitary formulation) such that their therapeutic efficacy overlaps.

The 5-HT_{1A} agonist may be employed in conjunction with an agent selected from the group consisting of stimulants, hypnotics, anxiolytics, antipsychotics, antianxiety agents, minor tranquilizers, benzodiazepines, barbituates, serotonin agonists, selective serotonin reuptake inhibitors, dopamine antagonists, 5-HT_{1A} agonists, 5-HT₂ antagonists, non-steroidal anti-inflammatory drugs, monoamine oxidase inhibitors, muscarinic agonists, norephinephrine uptake inhibitors, essential fatty acids, and neurokinin-1 receptor antagonist.

For example, for treating attention deficit disorder in a patient a 5-HT_{1A} agonist may be given in combination with such compounds as: adinazolam, allobarbital, alonimid, alprazolam, amitriptyline, amobarbital, amoxapine, bentazepam, benzoctamine, brotizolam, bupropion, busprione, butabarbital, butalbital, caffeine, capuride, carbocloral, chloral betaine, chloral hydrate, chlordiazepoxide, clomipramine, cloperidone, clorazepate, clorethate, clozapine, cyprazepam, deanol, desipranune, dexclamol, dextroamphetamine, diazepam, dichloralphenazone, divalproex, diphenhydramine, doxepin, duloxetine, estazolam, ethchlorvynol, etomidate, fenobam, flunitrazepam, flurazepam, fluvoxamine, fluoxetine, fosazepam, glutethimide, halazepam, hydroxyzine, imipramine, lithium, lorazepam, lormetazepam, maprotiline, mecloqualone, melatonin, mephobarbital, meprobamate, methaqualone, methylphenidate (including d-methylphenidate, especially d-methylphenidate hydrochloride), midaflur, midazolam, nefazodone, nisobamate, nitrazepam, nortriptyline, omega-3 fatty acids, oxazepam, paraldehyde, paroxetine, pemoline, pentobarbital, perlapine, perphenazine, phenelzine, phenobarbital, prazepam, promethazine, propofol, protriptyline, quazepam, reclazepam, roletamide, secobarbital, sertraline, suproclone, temazepam, thioridazine, tracazolate, tranylcypromaine, trazodone, triazolam, trepipam, tricetamide, triclofos, trifluoperazine, trimetozine, trimipramine, uldazepam, valproate, venlafaxine, xanomeline, zaleplon, zolazepam, zolpidem, and salts thereof, and combinations thereof, and the like, as well as admixtures and combinations thereof.

As used herein, the term “therapeutically effective amount” refers to that amount of a compound or preparation of the present invention that successfully prevents or reduces the severity of symptoms associated with attention deficit disorder, with or without hyperactivity. This term also embraces the amount of a compound or preparation of the present invention that successfully prevents or reduces the severity of symptoms associated with attention deficit disorder, with or without hyperactivity, anxiety, depression, and/or sexual dysfunction when the patient also suffers from anxiety, depression, obesity, drug abuse/addiction, alcohol abuse, sleep disorders, TIC disorder, or behavioral/cognitive symptoms of Alzheimer's disease.

As used herein, the term “TIC disorder” refers to a one or more disorders, which include Tourette's Disorder, Chronic Motor or Vocal Tic Disorder, Transient Tic Disorder, and Tic Disorder Not Otherwise Specified. A more complete description of the symptoms and diagnostic criteria of TIC disorder is provided by DSM-IV (Diagnostic and Statistical Manual of Mental Disorders, 1994; 100-105), which is incorporated herein by reference.

It is contemplated that the therapeutically effective amount of a composition will depend on a number of factors, including by not limited to the age of the patient, immune status, race, and sex of the patient, and the severity of the condition/disease, and the past medical history of the patient, and always lies within the sound discretion of the administering physician. Generally, the total daily dose of the compounds of this invention administered to a patient in single or in divided doses can be in amounts, for example, 0.25-0.75 mg/kg of body weight/day of gepirone (approximately 15 mg/day), 0.003-0.06 mg/kg of body weight/day of flesinoxan (approximately 0.4 mg/day), and 0.5-3.0 mg/kg of body weight/day of adatanserin (approximately 120 mg/day). Single dose compositions may contain such amounts or submultiples thereof to make up the daily dose. In general, treatment regimens according to the present invention comprise administration to a patient in need of such treatment 0.25-0.75 mg/kg of body weight/day of gepirone (approximately 15 mg/day), 0.003-0.06 mg/kg of body weight/day of flesinoxan (approximately 0.4 mg/day), and 0.5-3.0 mg/kg of body weight/day of adatanserin (approximately 120 mg/day) in single or multiple doses. In addition, the total daily dose of the azapirone compounds, having no dopamine receptor activity (including ipsapirone and tandospirone), of this invention administered to a patient in need thereof, in single or in divided doses can be in amounts of 0.25-3.0 mg/kg of body weight/day.

Treatment regimens according to the present invention also include concurrently administering to a patient in need thereof mixtures, in single or divided doses, of two or more of the compounds of the present invention. When the compounds of the present invention are administered concurrently as mixtures, the therapeutically effective amount to be administered lies within the sound discretion of the administering physician; preferably, the compounds of the present invention may be administered to a patient in single or in divided doses in amounts of, for example, 0.25-0.75 mg/kg of body weight/day of gepirone (approximately 15 mg/day); 0.003-0.06 mg/kg of body weight/day of flesinoxan (approximately 0.4 mg/day), 0.5-3.0 mg/kg of body weight/day of adatanserin (approximately 120 mg/day), and 0.25-3.0 mg/kg of body weight/day of ipsapirone and tandospirone.

Alternatively, treatment regimens according to the present invention include sequentially administering to a patient in need thereof, in single or divided doses, two or more of the compounds of the present invention. An example of a sequential administration strategy includes administering a therapeutically effective amount of a first compound followed by, on the same day or a subsequent day, a single or divided dose of a therapeutically effective amount of one or more additional compounds. As used herein, the term “subsequent day” refers to any day ranging from the next day (>24 hours) to one week (≦168 hours) after administration of the previous compound. The term “same day” refers to any time frame ranging from immediately after administration of the previous compound to ≦24 hours after administration of the previous compound.

When the compounds of the present invention are administered sequentially as a part of a combination therapy, the therapeutically effective amount to be administered lies within the sound discretion of the administering physician; preferably, the compounds of the present invention may be administered to a patient in single or in divided doses in amounts of, for example, 0.25-0.75 mg/kg of body weight/day of gepirone (approximately 15 mg/day); 0.003-0.06 mg/kg of body weight/day of flesinoxan (approximately 0.4 mg/day), 0.5-3.0 mg/kg of body weight/day of adatanserin (approximately 120 mg/day), and 0.25-3.0 mg/kg of body weight/day of ipsapirone and tandospirone.

As used herein, the terms “treat”, “treating”, and “treatment” also embrace the terms alleviation and amelioration. In addition, it is also within the scope of the present invention to use the methods described and/or claimed herein for the prevention of attention deficit disorder, with or without hyperactivity, as well as the symptoms associated therewith. Moreover, the terms “treat”, “treating”, and “treatment” also may embrace prevention of attention deficit disorder.

Having generally described this invention, a further understanding can be obtained by reference to certain specific examples, which are provided herein for purposes of illustration only, and are not intended to be limiting unless otherwise specified.

### EXAMPLES

Attention Deficit/Hyperactivity Disorder can exist alone or comorbidity with other psychiatric disorders in children. In fact, ADHD is one of the most common comorbidities associated with other disorders. ADHD can exist in conjunction with TIC disorder, Anxiety disorders, and Depressive disorders.

### Example 1

### The benefit of 5-HT_{1A} Partial Agonists in ADHD Alone

Experiments to gain FDA approval for this indication would involve two well-controlled, well-designed trials of the test drug in patients suffering with ADHD. A typical study would involve 50-100 children allocated 50% to the test drug and 50% to placebo. Medication would be given daily for approximately 8 weeks. Assessment of severity of ADHD symptoms would be completed prior to drug treatment and at regular intervals throughout the 8 weeks. The measurements and ratings would be similar to those mentioned above. Appropriate statistical procedures would be applied to the results. The study would be similar to that conducted by Greenhill et al (Pediatrics 2002; 109:E39-52).

### Example 2

### The Benefit of 5-HT_{1A} Partial Agonists in Children with ADHD and TIC Disorder

The procedure here would be similar to Example 1. An experiment similar to that provided by Gadow et al (J Clin Psychopharm 2002; 22:267-274) for methylphenidate (RITALIN™) could also be utilized.

### Example 3

### The Benefit of 5-HT_{1A} Partial Agonists in Children with ADHD and Anxiety Symptoms

The procedure here would be similar to Example 1. An experiment similar to those provided by Taylor et al (Psychol Med 1987; 17:121-143) and/or Pliszka (J Am Acad Child Adolesc Psychiatry 1989; 28:882-7) could also be utilized.

### Example 4

### The Benefit of 5-HT_{1A} Partial Agonist in Children with ADHD and Depressive Symptoms

The procedure here would be similar to Example 1. Additional diagnoses of depression would be made by DSM-IV criteria, and rating scales would include the Hamilton Depression Rating Scale (M. Hamilton, J Neurol Neurosurg Psychiatry 1960; 23:56-62).

Numerous modifications and variations on the present invention are possible in light of the above teachings. It is therefore, to be understood that within the scope of the accompanying claims, the invention may be practiced otherwise than as specifically described herein.

1. A method of treating attention deficit disorder, or symptoms thereof, in a patient in need thereof comprising administering a therapeutically effective amount of a metabolite of adamantyl aryl piperazinyl carboxamide or heteroaryl piperazinyl carboxamide 5-HT_{1A} receptor agonist, or a pharmaceutically acceptable salt thereof.

2. The method of claim 1, wherein the attention deficit disorder in the patient is further associated with hyperactivity.

3. The method of claim 1, wherein the metabolite of adamantyl aryl piperazinyl carboxamide or heteroaryl piperazinyl carboxamide is a metabolite of adatanserin.

4. The method of claim 1, wherein the metabolite of adamantyl aryl piperazinyl carboxamide or heteroaryl piperazinyl carboxamide is administered in conjunction with at least one agent selected from the group consisting of a stimulant, a hypnotic, an anxiolytic, an antipsychotic, an antianxiety agent, a minor tranquilizer, a benzodiazepine, a barbituate, a serotonin agonist, a selective serotonin reuptake inhibitor, a dopamine antagonist, a 5-HT_{1A} agonist, a 5-HT₂ antagonist, a non-steroidal anti-inflammatory drug, a monoamine oxidase inhibitor, a muscarinic agonist, a norephinephrine uptake inhibitor, an essential fatty acid, and a neurokinin-1 receptor antagonist.

5. The method of claim 1, wherein the metabolite of adamantyl aryl piperazinyl carboxamide or heteroaryl piperazinyl carboxamide is administered with methylphenidate.

6. The method of claim 1, wherein the metabolite of adamantyl aryl piperazinyl carboxamide or heteroaryl piperazinyl carboxamide is administered with a pharmaceutically acceptable carrier.

7. The method of claim 1, wherein said administering is selected from the group consisting of oral, rectal, nasal, parenteral, intracistemal, intravaginal, intraperitoneal, sublingual, topical, and bucal.

8. The method of claim 7, wherein said administering is oral or parenteral.

9. The method of claim 1, wherein the patient in need thereof also suffers from one or more disorders selected from the group consisting of anxiety, depression, obesity, drug abuse/addiction, alcohol abuse, sleep disorders, TIC disorder, and behavioral/cognitive symptoms of Alzheimer's disease.

10. The method of claim 1, wherein the patient in need thereof also suffers from one or more disorders selected from the group consisting of anxiety, depression, and TIC disorder.

11. A method of treating attention deficit disorder, or symptoms thereof, in a patient in need thereof comprising administering a therapeutically effective amount of two or more compounds selected from the group consisting of gepirone, ipsapirone, tandospirone, flesinoxan, and a metabolite of adatanserin.

12. The method of claim 11, wherein the attention deficit disorder in the patient is further associated with hyperactivity.

13. The method of claim 11, wherein the compounds are administered with at least one agent selected from the group consisting of a stimulant, a hypnotic, an anxiolytic, an antipsychotic, an antianxiety agent, a minor tranquilizer, a benzodiazepine, a barbituate, a serotonin agonist, a selective serotonin reuptake inhibitor, a dopamine antagonist, a 5-HT_{1A} agonist, a 5-HT₂ antagonist, a non-steroidal anti-inflammatory drug, a monoamine oxidase inhibitor, a muscarinic agonist, a norephinephrine uptake inhibitor, an essential fatty acid, and a neurokinin-1 receptor antagonist.

14. The method of claim 11, wherein the compounds are administered with a pharmaceutically acceptable carrier.

15. The method of claim 11, wherein said administering is selected from the group consisting of oral, rectal, nasal, parenteral, intracistemal, intravaginal, intraperitoneal, sublingual, topical, and bucal.

16. The method of claim 15, wherein said administering is oral or parenteral.

17. The method of claim 11, wherein the patient in need thereof also suffers from one or more disorders selected from the group consisting of anxiety, depression, obesity, drug abuse/addiction, alcohol abuse, sleep disorders, TIC disorder, and behavioral/cognitive symptoms of Alzheimer's disease.

18. The method of claim 11, wherein the patient in need thereof also suffers from one or more disorders selected from the group consisting of anxiety, depression, and TIC disorder.

19. The method of claim 11, wherein said two or more compounds are administered concurrently.

20. The method of claim 11, wherein said two or more compounds are administered sequentially.

21. The method of claim 20, wherein said two or more compounds are administered on the same day.

22. The method of claim 20, wherein said two or more compounds are administered on subsequent days.

23. A method of treating anxiety, or symptoms thereof, in a patient in need thereof comprising administering a therapeutically effective amount of an adamantyl aryl piperazinyl carboxamide or heteroaryl piperazinyl carboxamide 5-HT_{1A} receptor agonist, or a pharmaceutically acceptable salt thereof.

24. The method of claim 23, wherein the adamantyl aryl piperazinyl carboxamide or heteroaryl piperazinyl carboxamide is adatanserin, or a metabolite thereof.

25. The method of claim 23, wherein the adamantyl aryl piperazinyl carboxamide or heteroaryl piperazinyl carboxamide is administered in conjunction with at least one agent selected from the group consisting of a stimulant, a hypnotic, an anxiolytic, an antipsychotic, an antianxiety agent, a minor tranquilizer, a benzodiazepine, a barbituate, a serotonin agonist, a selective serotonin reuptake inhibitor, a dopamine antagonist, a 5-HT_{1A} agonist, a 5-HT₂ antagonist, a non-steroidal anti-inflammatory drug, a monoamine oxidase inhibitor, a muscarinic agonist, a norephinephrine uptake inhibitor, an essential fatty acid, and a neurokinin-1 receptor antagonist.

26. The method of claim 23, wherein the adamantyl aryl piperazinyl carboxamide or heteroaryl piperazinyl carboxamide is administered with methylphenidate.

27. The method of claim 23, wherein the adamantyl aryl piperazinyl carboxamide or heteroaryl piperazinyl carboxamide is administered with a pharmaceutically acceptable carrier.

28. The method of claim 23, wherein said administering is selected from the group consisting of oral, rectal, nasal, parenteral, intracisternal, intravaginal, intraperitoneal, sublingual, topical, and bucal.

29. The method of claim 28, wherein said administering is oral or parenteral.

30. The method of claim 23, wherein the patient in need thereof also suffers from one or more disorders selected from the group consisting of depression, obesity, drug abuse/addiction, alcohol abuse, sleep disorders, TIC disorder, and behavioral/cognitive symptoms of Alzheimer's disease.

31. The method of claim 23, wherein the patient in need thereof also suffers from one or more disorders selected from the group consisting of depression, and TIC disorder.

32. A method of treating depression, or symptoms thereof, in a patient in need thereof comprising administering a therapeutically effective amount of an adamantyl aryl piperazinyl carboxamide or heteroaryl piperazinyl carboxamide 5-HT_{1A} receptor agonist, or a pharmaceutically acceptable salt thereof.

33. The method of claim 32, wherein the adamantyl aryl piperazinyl carboxamide or heteroaryl piperazinyl carboxamide is adatanserin, or a metabolite thereof.

34. The method of claim 32, wherein the adamantyl aryl piperazinyl carboxamide or heteroaryl piperazinyl carboxamide is administered in conjunction with at least one agent selected from the group consisting of a stimulant, a hypnotic, an anxiolytic, an antipsychotic, an antianxiety agent, a minor tranquilizer, a benzodiazepine, a barbituate, a serotonin agonist, a selective serotonin reuptake inhibitor, a dopamine antagonist, a 5-HT_{1A} agonist, a 5-HT₂ antagonist, a non-steroidal anti-inflammatory drug, a monoamine oxidase inhibitor, a muscarinic agonist, a norephinephrine uptake inhibitor, an essential fatty acid, and a neurokinin-1 receptor antagonist.

35. The method of claim 32, wherein the adamantyl aryl piperazinyl carboxamide or heteroaryl piperazinyl carboxamide is administered with methylphenidate.

36. The method of claim 32, wherein the adamantyl aryl piperazinyl carboxamide or heteroaryl piperazinyl carboxamide is administered with a pharmaceutically acceptable carrier.

37. The method of claim 32, wherein said administering is selected from the group consisting of oral, rectal, nasal, parenteral, intracisternal, intravaginal, intraperitoneal, sublingual, topical, and bucal.

38. The method of claim 37, wherein said administering is oral or parenteral.

39. The method of claim 32, wherein the therapeutically effective amount of the adamantyl aryl piperazinyl carboxamide or heteroaryl piperazinyl carboxamide is 0.003 to 0.06 mg per kg of body weight per day.

40. The method of claim 32, wherein the patient in need thereof also suffers from one or more disorders selected from the group consisting of obesity, drug abuse/addiction, alcohol abuse, sleep disorders, TIC disorder, and behavioral/cognitive symptoms of Alzheimer's disease.

41. The method of claim 32, wherein the patient in need thereof also suffers from TIC disorder.

42. A method of treating sexual dysfunction, or symptoms thereof, in a patient in need thereof comprising administering a therapeutically effective amount of an adamantyl aryl piperazinyl carboxamide or heteroaryl piperazinyl carboxamide 5-HT_{1A} receptor agonist, or a pharmaceutically acceptable salt thereof.

43. The method of claim 42, wherein the adamantyl aryl piperazinyl carboxamide or heteroaryl piperazinyl carboxamide is adatanserin, or a metabolite thereof.

44. The method of claim 42, wherein the adamantyl aryl piperazinyl carboxamide or heteroaryl piperazinyl carboxamide is administered in conjunction with at least one agent selected from the group consisting of a stimulant, a hypnotic, an anxiolytic, an antipsychotic, an antianxiety agent, a minor tranquilizer, a benzodiazepine, a barbituate, a serotonin agonist, a selective serotonin reuptake inhibitor, a dopamine antagonist, a 5-HT_{1A} agonist, a 5-HT₂ antagonist, a non-steroidal anti-inflammatory drug, a monoamine oxidase inhibitor, a muscarinic agonist, a norephinephrine uptake inhibitor, an essential fatty acid, and a neurokinin-1 receptor antagonist.

45. The method of claim 42, wherein the adamantyl aryl piperazinyl carboxamide or heteroaryl piperazinyl carboxamide is administered with methylphenidate.

46. The method of claim 42, wherein the adamantyl aryl piperazinyl carboxamide or heteroaryl piperazinyl carboxamide is administered with a pharmaceutically acceptable carrier.

47. The method of claim 42, wherein said administering is selected from the group consisting of oral, rectal, nasal, parenteral, intracisternal, intravaginal, intraperitoneal, sublingual, topical, and bucal.

48. The method of claim 47, wherein said administering is oral or parenteral.

49. The method of claim 42, wherein the patient in need thereof also suffers from one or more disorders selected from the group consisting of attention deficit disorder, depression, anxiety, obesity, drug abuse/addiction, alcohol abuse, sleep disorders, TIC disorder, and behavioral/cognitive symptoms of Alzheimer's disease.

50. The method of claim 42, wherein the patient in need thereof also suffers from one or more disorders selected from the group consisting of anxiety, depression, and TIC disorder.

51. A method of psychoses, or symptoms thereof, in a patient in need thereof comprising administering a therapeutically effective amount of an adamantyl aryl piperazinyl carboxamide or heteroaryl piperazinyl carboxamide 5-HT_{1A} receptor agonist, or a pharmaceutically acceptable salt thereof.

52. The method of claim 51, wherein the adamantyl aryl piperazinyl carboxamide or heteroaryl piperazinyl carboxamide is adatanserin, or a metabolite thereof.

53. The method of claim 51, wherein the adamantyl aryl piperazinyl carboxamide or heteroaryl piperazinyl carboxamide is administered in conjunction with at least one agent selected from the group consisting of a stimulant, a hypnotic, an anxiolytic, an antipsychotic, an antianxiety agent, a minor tranquilizer, a benzodiazepine, a barbituate, a serotonin agonist, a selective serotonin reuptake inhibitor, a dopamine antagonist, a 5-HT_{1A} agonist, a 5-HT₂ antagonist, a non-steroidal anti-inflammatory drug, a monoamine oxidase inhibitor, a muscarinic agonist, a norephinephrine uptake inhibitor, an essential fatty acid, and a neurokinin-1 receptor antagonist.

54. The method of claim 51, wherein the adamantyl aryl piperazinyl carboxamide or heteroaryl piperazinyl carboxamide is administered with methylphenidate.

55. The method of claim 51, wherein the adamantyl aryl piperazinyl carboxamide or heteroaryl piperazinyl carboxamide is administered with a pharmaceutically acceptable carrier.

56. The method of claim 51, wherein said administering is selected from the group consisting of oral, rectal, nasal, parenteral, intracisternal, intravaginal, intraperitoneal, sublingual, topical, and bucal.

57. The method of claim 56, wherein said administering is oral or parenteral.

58. The method of claim 51, wherein the patient in need thereof also suffers from one or more disorders selected from the group consisting of attention deficit disorder, depression, anxiety, obesity, drug abuse/addiction, alcohol abuse, sleep disorders, TIC disorder, and behavioral/cognitive symptoms of Alzheimer's disease.

59. The method of claim 51, wherein the patient in need thereof also suffers from one or more disorders selected from the group consisting of anxiety, depression, and TIC disorder.

60. A method of treating aggressive behavior, or symptoms thereof, in a patient in need thereof comprising administering a therapeutically effective amount of an adamantyl aryl piperazinyl carboxamide or heteroaryl piperazinyl carboxamide 5-HT_{1A} receptor agonist, or a pharmaceutically acceptable salt thereof.

61. The method of claim 60, wherein the adamantyl aryl piperazinyl carboxamide or heteroaryl piperazinyl carboxamide is adatanserin, or a metabolite thereof.

62. The method of claim 60, wherein the adamantyl aryl piperazinyl carboxamide or heteroaryl piperazinyl carboxamide is administered in conjunction with at least one agent selected from the group consisting of a stimulant, a hypnotic, an anxiolytic, an antipsychotic, an antianxiety agent, a minor tranquilizer, a benzodiazepine, a barbituate, a serotonin agonist, a selective serotonin reuptake inhibitor, a dopamine antagonist, a 5-HT_{1A} agonist, a 5-HT₂ antagonist, a non-steroidal anti-inflammatory drug, a monoamine oxidase inhibitor, a muscarinic agonist, a norephinephrine uptake inhibitor, an essential fatty acid, and a neurokinin-1 receptor antagonist.

63. The method of claim 60, wherein the adamantyl aryl piperazinyl carboxamide or heteroaryl piperazinyl carboxamide is administered with methylphenidate.

64. The method of claim 60, wherein the adamantyl aryl piperazinyl carboxamide or heteroaryl piperazinyl carboxamide is administered with a pharmaceutically acceptable carrier.

65. The method of claim 60, wherein said administering is selected from the group consisting of oral, rectal, nasal, parenteral, intracisternal, intravaginal, intraperitoneal, sublingual, topical, and bucal.

66. The method of claim 65, wherein said administering is oral or parenteral.

67. The method of claim 60, wherein the patient in need thereof also suffers from one or more disorders selected from the group consisting of attention deficit disorder, depression, anxiety, obesity, drug abuse/addiction, alcohol abuse, sleep disorders, TIC disorder, and behavioral/cognitive symptoms of Alzheimer's disease.

68. The method of claim 60, wherein the patient in need thereof also suffers from one or more disorders selected from the group consisting of anxiety, depression, and TIC disorder.

69. A method of treating alcohol dependence, or symptoms thereof, in a patient in need thereof comprising administering a therapeutically effective amount of an adamantyl aryl piperazinyl carboxamide or heteroaryl piperazinyl carboxamide 5-HT_{1A} receptor agonist, or a pharmaceutically acceptable salt thereof.

70. The method of claim 69, wherein the adamantyl aryl piperazinyl carboxamide or heteroaryl piperazinyl carboxamide is adatanserin, or a metabolite thereof.

71. The method of claim 69, wherein the adamantyl aryl piperazinyl carboxamide or heteroaryl piperazinyl carboxamide is administered in conjunction with at least one agent selected from the group consisting of a stimulant, a hypnotic, an anxiolytic, an antipsychotic, an antianxiety agent, a minor tranquilizer, a benzodiazepine, a barbituate, a serotonin agonist, a selective serotonin reuptake inhibitor, a dopamine antagonist, a 5-HT_{1A} agonist, a 5-HT₂ antagonist, a non-steroidal anti-inflammatory drug, a monoamine oxidase inhibitor, a muscarinic agonist, a norephinephrine uptake inhibitor, an essential fatty acid, and a neurokinin-1 receptor antagonist.

72. The method of claim 69, wherein the adamantyl aryl piperazinyl carboxamide or heteroaryl piperazinyl carboxamide is administered with methylphenidate.

73. The method of claim 69, wherein the adamantyl aryl piperazinyl carboxamide or heteroaryl piperazinyl carboxamide is administered with a pharmaceutically acceptable carrier.

74. The method of claim 69, wherein said administering is selected from the group consisting of oral, rectal, nasal, parenteral, intracistemal, intravaginal, intraperitoneal, sublingual, topical, and bucal.

75. The method of claim 74, wherein said administering is oral or parenteral.

76. The method of claim 69, wherein the patient in need thereof also suffers from one or more disorders selected from the group consisting of attention deficit disorder, depression, anxiety, obesity, drug abuse/addiction, alcohol abuse, sleep disorders, TIC disorder, and behavioral/cognitive symptoms of Alzheimer's disease.

77. The method of claim 69, wherein the patient in need thereof also suffers from one or more disorders selected from the group consisting of anxiety, depression, and TIC disorder.

78. A method of weight loss, or obesity or symptoms thereof, in a patient in need thereof comprising administering a therapeutically effective amount of an adamantyl aryl piperazinyl carboxamide or heteroaryl piperazinyl carboxamide 5-HT_{1A} receptor agonist, or a pharmaceutically acceptable salt thereof.

79. The method of claim 78, wherein the adamantyl aryl piperazinyl carboxamide or heteroaryl piperazinyl carboxamide is adatanserin, or a metabolite thereof.

80. The method of claim 78, wherein the adamantyl aryl piperazinyl carboxamide or heteroaryl piperazinyl carboxamide is administered in conjunction with at least one agent selected from the group consisting of a stimulant, a hypnotic, an anxiolytic, an antipsychotic, an antianxiety agent, a minor tranquilizer, a benzodiazepine, a barbituate, a serotonin agonist, a selective serotonin reuptake inhibitor, a dopamine antagonist, a 5-HT_{1A} agonist, a 5-HT₂ antagonist, a non-steroidal anti-inflammatory drug, a monoamine oxidase inhibitor, a muscarinic agonist, a norephinephrine uptake inhibitor, an essential fatty acid, and a neurokinin-1 receptor antagonist.

81. The method of claim 78, wherein the adamantyl aryl piperazinyl carboxamide or heteroaryl piperazinyl carboxamide is administered with methylphenidate.

82. The method of claim 78, wherein the adamantyl aryl piperazinyl carboxamide or heteroaryl piperazinyl carboxamide is administered with a pharmaceutically acceptable carrier.

83. The method of claim 78, wherein said administering is selected from the group consisting of oral, rectal, nasal, parenteral, intracistemal, intravaginal, intraperitoneal, sublingual, topical, and bucal.

84. The method of claim 83, wherein said administering is oral or parenteral.

85. The method of claim 78, wherein the patient in need thereof also suffers from one or more disorders selected from the group consisting of attention deficit disorder, depression, anxiety, obesity, drug abuse/addiction, alcohol abuse, sleep disorders, TIC disorder, and behavioral/cognitive symptoms of Alzheimer's disease.

86. The method of claim 78, wherein the patient in need thereof also suffers from one or more disorders selected from the group consisting of anxiety, depression, and TIC disorder.
